(19) United States
(12) Patent Application Publication
(10) Pub. No.: US 2016/0143564 A1
(43) Pub. Date: May 26, 2016

(54) MEASURING DEVICE AND MEASURING METHOD FOR NON-INVASIVE DETERMINATION OF THE D-GLUCOSE CONCENTRATION

(71) Applicant: SMS SWISS MEDICAL SENSOR AG, Baar (CH)

(72) Inventors: Rolf-Dieter KLEIN, Muenchen (DE); Mathias REICHL, Kelheim (DE); Dieter EBERT, Gottlieben (CH)

(21) Appl. No.: 14/787,382

(22) PCT Filed: Jun. 23, 2014
(86) PCT No.: PCT/EP2014/001700

§ 371 Date: Oct. 27, 2015

(30) Foreign Application Priority Data
Jun. 25, 2013 (DE) 10 2013 010 611.7

(51) Int. Cl. A61B 5/1455; A61B 5/145

(52) U.S. Cl. CPC A61B 5/1455; A61B 5/14532

(57) ABSTRACT

The invention relates to a measuring method and a measuring instrument for measuring raw data for determination of a blood parameter, in particular for non-invasive determination of the D-glucose concentration. The measuring device (1) comprises an excitation source (2) for generating electromagnetic radiation, a coupling arrangement (5-8) which is configured to couple in the radiation emitted by the excitation source (2) into a body surface of an object to be measured, and a sensor arrangement (13) which is configured to detect infrared (IR) radiation which is excited by the coupled-in radiation of the excitation source (2) in the body surface. The coupling arrangement (5-8) is configured to couple in the radiation emitted by the excitation source (2) extensively at a plurality of measuring points into the body surface, and the sensor arrangement (13) is configured to detect the IR radiation generated in the body surface at a plurality of measuring points.

The invention relates to a measuring method and a measuring device for measuring raw data for determining a blood parameter, particularly for the non-invasive determination of the D-glucose concentration.

Approaches for the non-invasive in vivo measurement of the blood sugar concentration are known from the prior art. Herein, for example, a tissue layer is optically stimulated in order to measure the blood sugar level from the measured absorption of the radiation which depends on the glucose concentration.

A disadvantage of known methods is that previously sufficient accuracy could not be achieved in the non-invasive determination of the glucose concentration, since the absorption at the known glucose absorption bands is overlaid with the strong absorption and scattering effects of other substances and tissue constituents, as illustrated, by way of example, in FIG. 5.

A non-invasive measuring approach of this type is known, for example, from U.S. Pat. No. 7,729,734 B2. A measuring device is proposed wherein two modulated laser beams of different wavelengths phase-offset by 180° are used for the excitation of the glucose. The phase-shifted laser beams generate two phase-shifted photothermal signals in the infrared (IR) region, which are detected with an infrared detector. The evaluation of a laser-induced wavelength-modulated phase-shifted signal enables a suppression of the strong background signal which is generated mainly by water. From the amplitude and the phase of the differential infrared signal detected, a conclusion can be drawn about the glucose concentration.

However, a disadvantage of the proposed measuring device is that the determination of the glucose concentration from the measured IR signal requires a phase resolution of 0.1° which cannot be achieved under practical usage conditions.

It is therefore an object of the invention to avoid the disadvantages of known non-invasive measuring devices. The measuring device should, in particular, be able to generate raw data which enable an accurate, non-invasive determination of a blood parameter, in particular the D-glucose concentration. It is a further object to provide a measuring method for measuring raw data for determining a blood parameter, particularly for the non-invasive determination of the D-glucose concentration with which disadvantages of conventional methods can be avoided.

These objects are achieved by means of a measuring device and a measuring method having the features of the independent claims. Advantageous embodiments and uses of the invention are disclosed by the dependent claims.

The invention is based on the recognition that the disruptive erroneous measurements in the known measuring device can be caused in that the measurements can be influenced by locally restricted irregularities of the irradiated body surface or the skin layers lying thereunder, for example, by means of pimples, adipose veins, bones, cornified skin layers, sweat and/or thickness variations of the capillary vessels.

The invention therefore includes the general technical teaching of carrying out the measurement not only at a single measurement position on the body surface of the test object under investigation, but at a plurality of measurement positions. The measurement positions can be configured as spaced-apart or overlapping measurement regions or as measurement points spaced apart from one another.

The measuring device according to the invention has, in agreement with the prior art, at least one excitation source for generating electromagnetic radiation and a coupling-in apparatus in order to couple the radiation emitted by the excitation source into a body surface of a measurement object.

The measuring device also comprises a sensor apparatus in order to detect the infrared (IR) radiation which is stimulated in the body surface by the coupled-in radiation of the excitation source.

In order to carry out the measurement not only at one measurement point, the coupling-in apparatus is configured to couple the radiation emitted by the excitation source areally at a plurality of measurement positions into the body surface and the sensor apparatus is configured to detect the IR radiation generated in the body surface at a plurality of measurement positions.

The use according to the invention of a plurality of measurement positions has the advantage that erroneous measurement points are recognized in the context of the above-mentioned locally delimited irregularities and can be compensated for by suitable selection of the measurement positions. When measuring the stimulated IR radiation at only one measurement position, it is not possible to differentiate whether the measured intensity value of the IR radiation was generated by a particular glucose concentration X or by a glucose concentration Y, wherein the IR intensity generated was additionally influenced by a local disturbing effect, for example, a thicker than average cornified skin layer. In the case of a plurality of measurement positions, by means, for example, of mean value formation across all the measurement positions, those whose deviations from the mean value is greater than a pre-determined threshold value can be identified as “erroneous” since the measurement value has probably been influenced by local irregularities and/or undesired disturbing effects. In this way, the sorting out of the “erroneous” measurement positions improves the measurement accuracy. Furthermore, by mean value formation on the basis of the remaining “non-erroneous” measurement positions, an improvement in the accuracy of the determination of the blood parameter value, particularly the D-glucose concentration, can be achieved, since variations in the background signals can be averaged out by this means.

The coupling-in apparatus is preferably configured so that the measurement positions are arranged on the skin surface at regular spacings, for example, in a grid pattern. The measurement positions can however also be arranged irregularly.

Preferably, the measurement positions at which the electromagnetic radiation is coupled-in match the measurement positions at which the stimulated IR radiation is detected. However, the possibility also exists that the measurement positions for coupling-in the electromagnetic radiation and the measurement positions for detecting the IR radiation are slightly offset relative to one another or differ slightly in their number, provided the generated electromagnetic radiation is coupled in areally and the generated IR signal can be read out areally.

According to a particularly preferred embodiment of the invention, the excitation source is a tuneable excitation source for generating electromagnetic radiation in the visible and/or IR range. According to this variant, the measuring device is configured to tune the excitation source across a pre-determined spectral region during a measuring procedure.

This has the advantage that the measurement data are based not only on one or two point(s) of the glucose absorption spectrum, but that a spectral sequence over a pre-determined frequency range can be used for determining the blood sugar concentration.

This is based on the discovery by the inventors that disturbing effects caused, for example, by a high level of water absorption or by other components, can be reliably identified by means of the recording of a tuned IR spectrum. Thus, for example, by means of a correlation analysis, the agreement of the recorded measurement curve with a reference spectrum can be determined. Measured IR intensity curves which have a high level of agreement in the tuned frequency region with the reference curve, e.g. the D-glucose absorption curve, show that the coupled-in radiation was absorbed at the measurement position by glucose molecules, whereas a low level of agreement with the reference curve shows that the coupled-in radiation was absorbed or scattered by water or other substances.

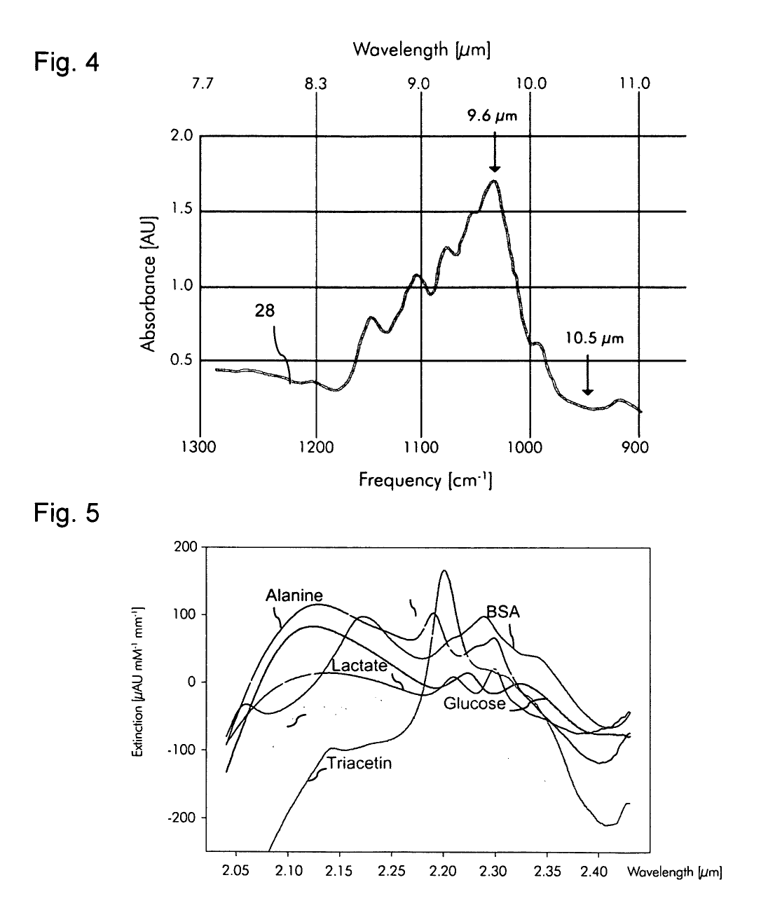

A particular advantage of the invention therefore lies therein that only the measurement curves which have also actually measured the glucose absorption for which the absorption curve is shown by way of example in FIG. 4 and have not been falsified by absorption curves of other components, which are shown by way of example in FIG. 5, can be used for determining the D-glucose concentration. By contrast therewith, the approaches known from the prior art which measure only one or two fixed wavelengths cannot reliably differentiate whether a measured value shows, for example, a raised glucose concentration or rather was merely falsified by means of an adjacent absorption curve of another substance.

According to a further embodiment, the coupling-in apparatus can comprise a scanning unit which is configured to irradiate the plurality of measurement positions on the coupling-in surface time-sequentially (by “flying spot irradiation”). The coupling-in apparatus can be configured, for example, as a micro-scanner or a MEMS scanner or can comprise a Digital Light Processing (DLP) unit.

This has the advantage that the energy density at the respectively irradiated measurement position on coupling-in of the excitation beams is increased and thus the penetration depth, but without increasing the mean value of the energy density over the whole coupling-in area.

The sensor apparatus for areally detecting the IR radiation emerging at the plurality of measurement positions is preferably an infrared area sensor, for example, an IR-CCD sensor.

Advantageously the IR radiation generated at each of the different measurement positions is imaged on a different region of the IR area sensor. For example, measurement positions arranged in a grid shape at which the IR radiation generated in the skin layer is detected can be imaged on a corresponding grid structure in the form of columns and rows of an area sensor so that the positional information of the measurement points is retained.

By a comparison of the measurement values of all the measurement positions, positional errors, that is the measurement positions which are unsuitable for the determination of the blood parameter, can be identified and correspondingly left out of consideration in the further processing of the measured raw data.

The measuring device according to the invention generates raw data in the form of the measured intensities of the IR radiation, preferably resolved according to the position of the measurement position and the wavelength of the tuneable excitation source. On the basis of these measurement data, by means of subsequent data processing, a D-glucose concentration or, in general, the value of a blood parameter can be determined.

For this purpose, the measuring device can comprise an evaluation unit which determines the blood parameter value, for example, the blood sugar concentration, depending on the detected IR radiation and on the stored reference spectra.

A particularly advantageous use of the measuring device according to the invention is the measurement of raw data in order, on the basis of the raw data, to determine an in-vivo D-glucose concentration. In the following section, reference is repeatedly made to this use of the measuring device according to the invention, emphasized by way of example. It is emphasized that the present invention can also be generalized to the effect that the measuring device can be used for determining other blood parameters, for example by storing suitable reference spectra for these blood parameters and by selection of a suitable frequency range which is adapted to the absorption curve of this blood parameter.

The evaluation unit is preferably arranged to compare the detected IR data with previously determined reference spectra. For the creation of reference spectra, IR measurements are carried out by the measuring device and correlated with the D-glucose concentration which is determined, for example, with conventional invasive methods.

In an advantageous embodiment of the invention, in order to measure an intensity sequence in the tuned spectral sequence, the detected IR radiation is imaged for each of the measurement positions on a different region of the IR area sensor. In other words, a 1:1 mapping of a measurement position to a subregion of the area sensor takes place, so that the measurement region is imaged in two-dimensional resolution on the area sensor, for example, characterized by a particular column and row of the area sensor.

Each subregion of the area sensor which corresponds to a particular measurement position then records an excitation spectrum for the corresponding measurement position on tuning of the excitation source. The evaluation unit can then identify, as described above, by comparing with reference spectra and/or by mean value formation, those measurement positions which are suitable or unsuitable for determining the blood parameter value.

Another possibility of realization according to the invention provides that the sensor apparatus has a spectrometer. For example, the sensor apparatus can comprise an optical grating or prism which is configured to image different wavelength ranges of the IR radiation generated in the body surface onto different columns of the IR area sensor. The rows of the IR area sensor are each associated with a group of measurement positions on the body surface. Herein, the definition as to which of the two planar extent directions of the area sensor constitutes columns and which constitutes rows is arbitrary.

According to this variant, the evaluation unit is configured to identify, by comparison with reference spectra and/or by mean value formation, those rows whose detected IR intensity values are suitable for determining the D-glucose concentration. By contrast, those rows in which disturbing effect are recognized can be separated out. By this means, the accuracy of the blood sugar measurement can be further increased.

In this embodiment variant, the spatial resolution is reduced by one dimension, since one dimension of the area sensor is used for the spectral resolution of the IR signal. A particular advantage of this exemplary embodiment, however, is that valuable information can be obtained from the spectral decomposition of the detected IR signal, in order to increase the accuracy of the D-glucose determination. For example, additional fluorescence effects can be measured or a plurality of peaks of the glucose absorption curve can be measured at an excitation frequency.

A D-glucose concentration correlates, for example, with the height of a glucose absorption peak. If additional fluorescence effects now occur, the measured height of the glucose absorption peak can be changed thereby—with an unchanged glucose concentration. By means of the additional spectral decomposition, such effects can be recognized and taken into account in the form of a correction factor.

It has already been mentioned above that, by means of locally delimited irregularities of the skin surface structure and generally, due to the low concentration of the glucose molecules as compared with water and other components in the capillary blood, undesirable falsification of the measurement data can occur.

The evaluation unit therefore preferably determines the peaks of the detected IR rays, the wavelengths of each peak and/or the intensity of each peak.

Subsequently, the evaluation unit then preferably determines the intensity ratio of the peaks, that is, for example, the ratio of the intensity of the first peak and the intensity of the second peak, so that the main absorption peaks and any existing subsidiary peaks can be determined.

Furthermore, the evaluation unit preferably determines the wavelength match between the wavelengths of the measured peaks of the IR radiation and the pre-determined characteristic wavelengths that are characteristic for the glucose absorption. The intensity ratio of the measured peaks and the wavelength match of the measured peaks with the characteristic wavelengths then enable an assessment of whether the measured radiation actually originates from the glucose absorption or from disturbances.

The evaluation unit can also determine the intensity ratio of a peak of the detected IR radiation to a corresponding peak of a pre-determined reference curve or can compare the peak of the detected IR radiation with a corresponding peak of the pre-determined reference curve for determination of the glucose concentration.

Furthermore, the evaluation unit can carry out the mean value formation described above over the individual pixels or rows of the sensor in order to identify the measurement positions and measurement curves influenced by disturbing effects.

Furthermore, the signal emitted by the excitation source can be modulated. In this case, the evaluation unit is configured to determine a dispersion angle, depending on the modulated signal. Herein, the longer-wavelength carrier signal, preferably in the infrared region, ensures that the desired depth of penetration into the upper skin layers is reached, whilst the modulated-on signal additionally enables the evaluation of the dispersion angle.

The excitation source can be a tuneable quantum cascade laser. The wavelength of the radiation generated by the excitation source preferably lies in the range from 250 nm to 30 μm. Further preferably, the radiation generated can lie in the range from 7 μm to 14 μm, in which there is a distinct glucose absorption band from 8.5 μm to 10.5 μm, with a peak at approximately 9.6 μm.

The tuneable excitation source is preferably tuned in a pre-determined spectral region which comprises one or more peaks in the D-glucose absorption band, preferably in the IR region, since in this region, the glucose absorption bands are sufficiently distinct and the penetration depth of the coupled-in radiation is sufficient to reach the capillary vessels at 1.5 μm to 2 μm depth.

The measurement accuracy can be further improved in the context of the invention if the sensor apparatus comprises, apart from the IR area sensor, a further IR photodiode which detects an infrared radiation which is stimulated by the coupled-in radiation of the excitation source in the body surface. The IR radiation detected over the whole area of the measurement positions is measured by the photodiode as a mean value.

The IR photodiode can be used for temperature measurement for correcting a temperature-related variation of the IR signal detected at the measurement positions or for forming a reference signal in order to correct any scattering effects occurring.

Furthermore, the evaluation unit can be configured to monitor the current output of the tuneable excitation source and to regulate the excitation source during tuning such that it remains constant or has a pre-determined curve shape. Since the power regulation of a laser is dependent, for example, on the wavelength, the measurement accuracy can be further increased by means of this additional regulating circuit, since the intensity of the detected IR radiation can be normalized depending on the laser output.

Preferably, the coupling-in apparatus comprises a measuring head, the form of which is adapted to an upper or lower fingertip, a heel and/or an ear lobe of the test object. For this purpose, the measuring head can have a planar or curved contact surface or can also be configured as a clip. In order to prevent errors due to false positioning on the measuring surface, the coupling-in apparatus can be further configured to determine, before the execution of a measuring procedure, whether a lower or upper fingertip, a heel or an ear lobe of the test object is positioned in a pre-determined region on the measuring head.

The light emitted by the excitation source can be coupled areally into the body surface by means of an optical fiber bundle or an optical unit. In the embodiment without a grating or spectrometer, the IR radiation detected can also be imaged directly by means of an optical fiber bundle onto the corresponding regions of the IR area sensor. In the embodiment with a spectrometer or with an optical grating or prism, however, an additional optical unit is provided in order to form the optical intensity mean value of the measurement positions of a measuring line, which is then spectrally decomposed by the optical grating and is imaged onto a row of the area sensor.

The invention further relates to a method for measuring raw data for determining a blood parameter, particularly for the non-invasive determination of the D-glucose concentration, comprising the steps: generating electromagnetic radiation, coupling the generated radiation into a body surface of a measurement object and detecting an infrared radiation which is stimulated in the body surface by means of the coupled-in radiation, wherein the radiation generated is coupled into the body surface areally at a plurality of measurement positions. Preferably, the coupled-in electromagnetic radiation is tuned during a measuring procedure across a pre-determined spectral region in the visible and/or the IR range.

The previously described aspects of the measuring device can also be configured as corresponding method steps without this being explicitly stated.

Further details and advantages of the invention will now be described making reference to the accompanying drawings, in which:

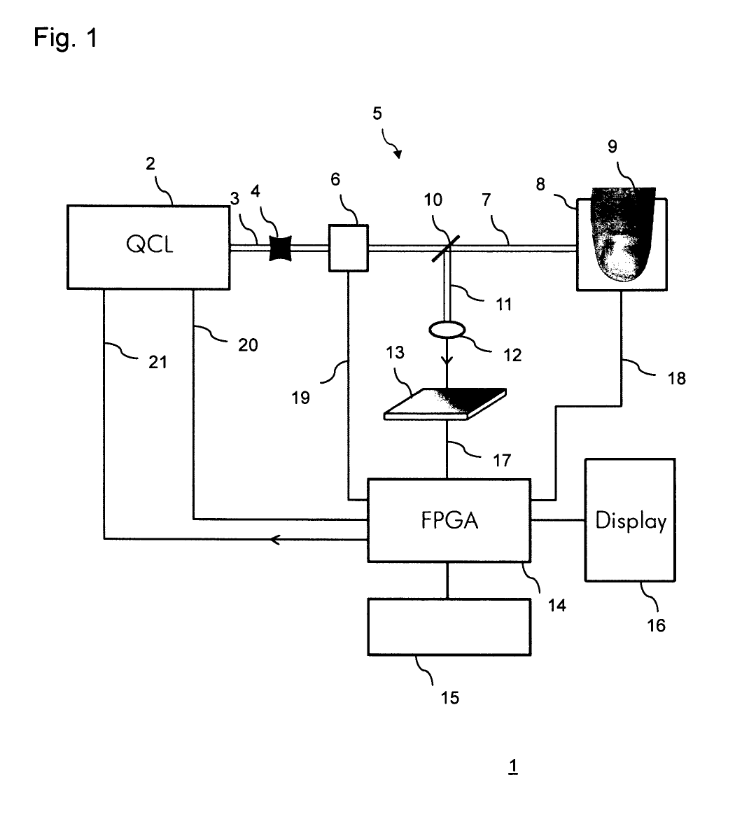

FIG. 1 shows a schematic block circuit diagram of a measuring device according to one exemplary embodiment;

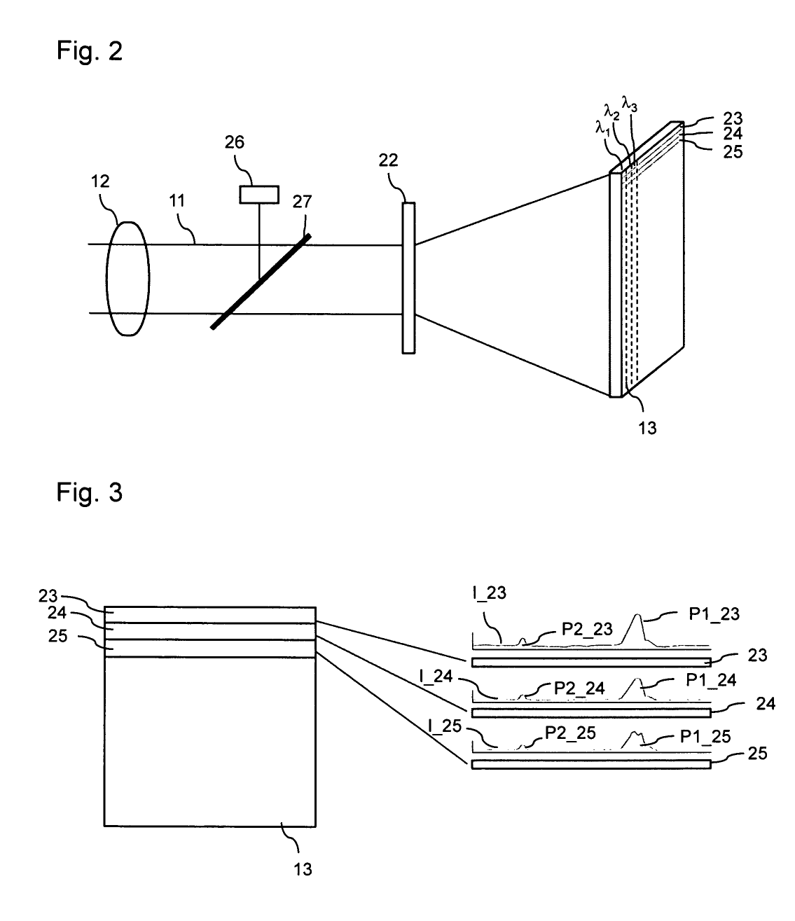

FIG. 2 shows a sensor apparatus of a measuring device according to the invention according to a further exemplary embodiment;

FIG. 3 shows the spectral decomposition of the detected IR signal on the IR area sensor according to an exemplary embodiment;

FIG. 4 shows a main absorption peak of glucose in the IR range;

FIG. 5 shows the glucose absorption curve compared with absorption curves of other components present in the blood; and

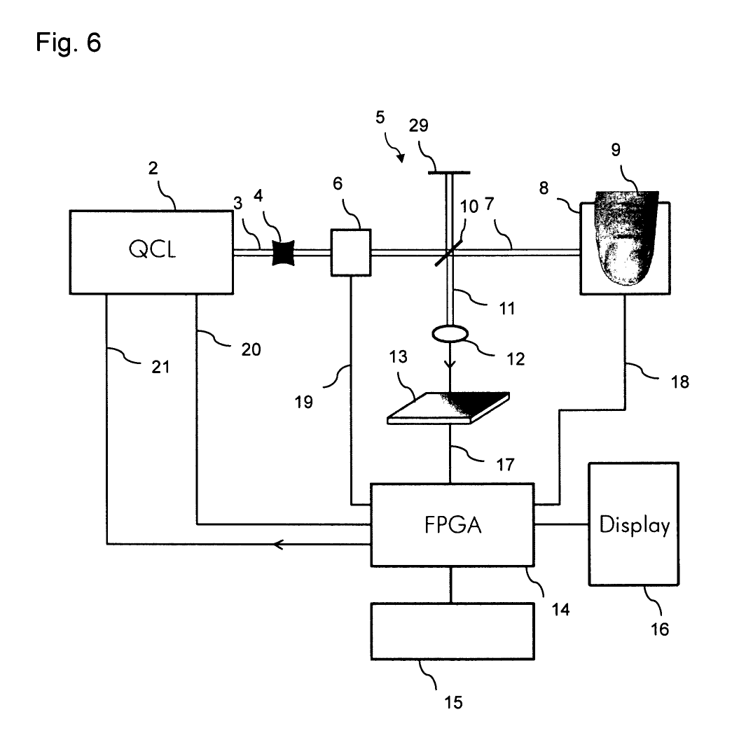

FIG. 6 shows a schematic block circuit diagram of a measuring device according to a further exemplary embodiment.

FIG. 1 shows a schematic block circuit diagram of a measuring device 1 according to the invention for non-invasive determination of the D-glucose concentration.

For the non-invasive determination of the D-glucose concentration, the person whose blood sugar concentration is to be measured places a lower fingertip 9 on the measuring surface of a measuring head 8. In the present example, the measuring surface is configured as a planar contact surface. The blood sugar concentration can, however, also advantageously be measured at the upper fingertip or the heel or an earlobe, since capillary vessels lie there at a shallow penetration depth. For placement on the upper fingertip or the heel, the measuring surface can also be curved in order to adapt the measuring surface to the surface form of the body site to be measured.

The measuring device 1 comprises a quantum cascade laser 2 which is tuneable in a pre-determined wavelength range. According to the exemplary embodiment described, the measuring device 1 is configured to tune the quantum cascade laser 2 for a measuring procedure in the range from 7 μm to 14 μm. In this range, there is a main absorption band of glucose, as shown in FIG. 4. The band extends from 8.8 μm to 10.5 μm and has a peak at approximately 9.6 μm. Herein, the clocking when passing through the pre-determined frequencies or frequency intervals of the frequency range can lie in the range from 0.1 Hz to 12 kHz.

The laser light output by the quantum cascade laser 2 is conducted by means of an optical fiber conductor 3 and a suitable generic optical unit 4 to a coupling-in apparatus 5. The coupling-in apparatus 5 couples the radiation emitted by the excitation source 2 areally into the lower fingertip 9.

In the present example, the coupling-in apparatus 5 consists of a microscanner 6, an optical fiber bundle 7 wherein each optical fiber ends in the immediate vicinity of a measurement point, and the measuring head 8.

The individual measurement points are arranged in a grid form in rows and columns (not shown). The microscanner 6 controls the points arranged in a grid shape one after the other, for example row by row, which is also known as a flying spot process. The laser light radiated in is absorbed in the upper skin layers of the lower fingertip 9 and is output again as infrared radiation. The infrared radiation generated by the coupled-in laser radiation is imaged by means of a suitable generic optical unit 12 onto an IR area sensor 13 which is an IR-CCD sensor.

Herein, each of the plurality of measurement positions is imaged on a pre-determined region on the IR-CCD sensor 13, so that a 1:1 association with the corresponding positions or pixels on the sensor surface takes place.

Thus, the position information of the measurement positions is retained and enables a geometric evaluation of the individual measurement positions, i.e. an evaluation of the measurement positions according to their position on the skin surface.

The elements identified with the reference signs 4, 10, and 12 represent generic optical elements such as beam splitters, lenses, mirrors, etc. which are per se known from the prior art and form the beam path for the excitation beam or the beam path for the detected IR radiation.

For each frequency or for each frequency interval of the frequency range passed through, a measurement value is recorded by the area sensor 13, so that the measuring device 1 measures, for each of the plurality of measurement points, a measurement series for each frequency interval passed through in the form of the intensity of the detected IR radiation.

For the evaluation of the measurement data and for controlling the measuring procedure, a central evaluation and control unit 14 is provided which can be realized, for example, as a Field Programmable Gate Array (FPGA).

The control unit 14 controls and synchronizes the laser 2, the scanning unit 6, the area sensor 12 and the measuring head 8 by means of signal lines 17-21. The control unit 14 receives the data measured by the area sensor 13 by means of a signal line 17. The control unit 14 is connected to the measuring head 8 by means of a further signal line 18, by means of which the measuring head 8 signals to the control unit 14 whether a fingertip 9 has been positioned on the measuring head 8 and whether it has been correctly positioned, i.e. in a pre-determined region of the contact surface. If this is the case, the control unit 14 carries out the measuring procedure, otherwise a warning signal is output.

The signal lines 20 and 21 are part of a control loop for controlling the laser 2 by means of the evaluation unit 14. By means of the control line 21, the evaluation unit 14 can control the tuneable cascade laser 2 such that when performing a measuring procedure, the laser passes through a pre-determined frequency range with a particular clock cycle. Furthermore, the output of the laser 2 can be regulated. Since the output of the laser 2 varies with the wavelength, by means of a decoupling, the output of the laser 2 is communicated via the signal line 20 to the control unit 14 by means of the signal line 20 and is monitored by the control unit. In this way, an intensity variation of the laser source 2 can be avoided to prevent a change in the measured intensity of the IR radiation being influenced also by the variation of the laser intensity. Alternatively, the detected IR signal can be normalized depending upon the measured laser intensity.

By means of a further signal line 19, the evaluation unit 14 controls the microscanner 6 during the performance of a measuring procedure. Furthermore, the measurement data determined by the measuring device 1 can be shown on a display 16. Previously determined reference spectra are stored in a storage unit 15.

For each frequency interval of the excitation source that is passed through, the evaluation unit 14 reads out the intensities of the stimulated IR radiation detected by the infrared area sensor 13, so that for each of the plurality of measurement points, an intensity sequence is measured over the stimulated wavelengths. Herefrom, the evaluation unit 14 can determine for each measurement point the peaks of the detected IR radiation and the intensity of each peak. The expression peak in the context of this invention also includes an absorption peak at which the measured intensity of the detected IR radiation decreases.

In this way, the evaluation unit 14 can determine, for example, the position and height of the main absorption peak at 9.6 μm, as shown in FIG. 4.

By comparison of the measured value with the reference curves stored in the memory unit 15, the glucose concentration can be determined.

In order to improve the measurement accuracy, the evaluation unit 14 compares the individual measurement series of all the measurement points. For this purpose, for example, the variations of the measured intensity values of the IR radiation for each wavelength range passed through can be compared over the individual measurement positions. This can be carried out, for example, by mean value formation across all the measurement positions and subsequent determination of the deviation of each measurement position from the mean value. By this means, positional errors and spectral errors can be identified and calculated out. If the measured intensity of the IR radiation at a measurement position or at a plurality of measurement positions deviates strongly as compared with the majority of measurement positions, the corresponding measurement values are not taken into account during the determination of the D-glucose concentration.

FIG. 2 is a further exemplary embodiment of the present invention. Herein, only the sensor apparatus of the measuring device 1 is shown enlarged, since the other components correspond to those of FIG. 1.

As distinct from the measuring device of FIG. 1, the sensor apparatus of FIG. 2 comprises an additional optical grating 22 which is configured to image different wavelength ranges of the IR radiation generated in the body surface onto different columns of the IR area sensor 13. Therefore, a spectral decomposition of the IR spectrum detected takes place such that the rows of the IR area sensor 13 are each associated with different measurement positions on the body surface, whilst the different wavelength ranges are imaged on different columns of the area sensor 13.

This is shown, by way of example, in FIGS. 2 and 3 for the first three rows 23, 24 and 25 of the area sensor 13.

All the measurement values of a particular column correspond to an intensity of the IR signal which was measured at a wavelength or a wavelength range. Each of the pixels of the first column corresponds to a detected IR intensity value at the wavelength Δ1, those of the second column at the wavelength Δ2, those of the third column at the wavelength Δ3, etc.

Due to the additional spectral splitting with the optical grating 22, the measurement positions can no longer be two-dimensionally spatially resolved on the area sensor 13 as in the exemplary embodiment of FIG. 1. Therefore, the measured intensity values of the individual measurement positions of a row of the measurement area are optically averaged before they meet the grating 22 and are imaged spectrally decomposed by said grating onto a corresponding row of the area sensor 13 (not shown).

The curve I_23 in FIG. 3 corresponds to a measured IR spectrum of the first row 23 of the area sensor 13 and shows a main peak P1_23 which corresponds to the absorption peak at 9.6 μm and a second peak P2_23 at approximately 5 μm, which corresponds to a fluorescence peak.

In the simplified representation of FIG. 3, the absorption peak P1_23 is also shown heightened, although this corresponds to a reduction in the detected intensity.

The detected intensity spectra I_24 and I_25 of rows 24 and 25 show a similar, but not exactly identical shape to the spectrum of the first row 23.

The evaluation unit 14 can now analyze the individual peaks of the detected absorption spectrum. Herein, rows with excessively severe deviations from the mean value over all the rows are separated out as erroneous measurements, so as to increase the accuracy of the determination of the glucose concentration. Furthermore, rows which have a measured peak structure, for example, number and height ratios of the peaks which severely deviate from the peak structure of a reference curve can be identified as local erroneous measurements. The peak structures can be compared by means of a correlation analysis.

If, for example, a row contains a fluorescence peak at a wavelength of 8 μm instead of the expected 5 μm, the evaluation unit can classify the corresponding measurement series as a measurement error and leave the data out of consideration during the determination of the glucose concentration.

The sensor device according to the exemplary embodiment of FIG. 2 comprises a further IR photodiode 26 which measures the infrared radiation over the entire area of the measurement positions as a mean value. For this purpose, the IR radiation measured by the measurement positions is guided by means of a beam splitter 27 to the photodiode 26. The IR photodiode 26 is used for temperature measurement for correcting a temperature-related variation of the IR signal detected at the measurement positions.

FIG. 6 shows a schematic block circuit diagram of a measuring device according to a further exemplary embodiment. According to this exemplary embodiment, the signal emitted by the excitation source 2 is modulated. Herein, the longer-wavelength carrier signal, preferably in the infrared region, ensures that the desired penetration depth into the upper skin layers is achieved, whilst the modulated-on signal additionally enables the evaluation of the dispersion angle.

As distinct from the exemplary embodiment of FIG. 1, the measuring device comprises a further mirror 29 in order to be able to carry out an interferometric measurement. Herein, a part of the laser light coming from the laser source 2 is guided through the semitransparent mirror 10 onto the mirror 29, reflected there and then imaged perpendicularly on the area sensor 13. However, a part of the laser light coming from the laser source 2 is coupled into the fingertip 9. The radiation stimulated in the fingertip 9 is guided back to the mirror 10 and is also imaged thereby on the area sensor 13. These two light beams imaged on the area sensor 13 interfere on the area sensor 13 to form an interference pattern (not shown). From the interference pattern, a refractive index can be calculated which has a characteristic value for glucose. With this additional measurement variant, the measurement accuracy can be further increased.

The features of the invention disclosed in the present description, the drawings and the claims can be significant either individually or in combination for the realization of the invention in its various embodiments.

1-15. (canceled)

16. A measuring device adapted for measuring raw data for determining a blood parameter, comprising
a) at least one excitation source for generating electromagnetic radiation,
b) a coupling-in apparatus which is configured to couple the electromagnetic radiation emitted by the at least one excitation source into a body surface of a measurement object, and
c) a sensor apparatus which is configured to detect an infrared (IR) radiation which is stimulated in the body surface by the electromagnetic radiation coupled-in to the body surface, wherein
d) the coupling-in apparatus is configured to couple into the body surface the electromagnetic radiation emitted by the at least one excitation source areally at a plurality of measurement positions, and
e) the sensor apparatus is configured to detect the IR radiation generated in the body surface at a plurality of measurement positions.

17. The measuring device according to claim 16, wherein the at least one excitation source is a tuneable excitation source for generating electromagnetic radiation in at least one of the visible or IR range and the measuring device is configured to tune the at least one excitation source across a pre-determined spectral range during a measuring procedure.

18. The measuring device according to claim 16, wherein the coupling-in apparatus comprises a scanning unit which is configured to irradiate the plurality of measurement positions of the body surface time-sequentially.

19. The measuring device according to claim 17, wherein the sensor apparatus comprises an IR area sensor for detecting the IR radiation emitted at the plurality of measurement positions.

20. The measuring device according to claim 19, wherein the IR radiation generated at each measurement position of the plurality of measurement positions is imaged on a different region of the IR area sensor.

21. The measuring device according to claim 16, further comprising an evaluation unit for determining a blood parameter value depending on the IR radiation detected and on stored reference spectra.

22. The measuring device according to claim 19, wherein during tuning of the at least one excitation source across the pre-determined spectral range, the IR radiation detected is imaged for each of the plurality of measurement positions on a different region of the IR area sensor in order to measure an intensity sequence in a tuned spectral sequence.

23. The measuring device according to claim 21, wherein the evaluation unit is configured to identify, by at least one of comparison with reference spectra or mean value formation, those measurement positions which are suitable for determining a D-glucose concentration.

24. The measuring device according to claim 21, wherein the sensor apparatus comprises at least one of a spectrometer or an optical grating which is configured to image different wavelength ranges of the IR radiation generated in the body surface onto different columns of an IR area sensor, wherein the rows of the IR area sensor are each associated with different measurement positions on the body surface.

25. The measuring device according to claim 24, wherein the evaluation unit is configured to identify, by at least one of comparison with reference spectra or mean value formation, those rows whose detected IR intensity values are suitable for determining the D-glucose concentration.

26. The measuring device according to claim 21, wherein the evaluation unit determines at least one of:
a) from the peaks of the IR radiation detected, the wavelength of each peak or the intensity of each peak,
b) the intensity ratio of a peak of the IR radiation detected to a corresponding peak of a pre-determined reference curve, or
c) a wavelength match between, firstly, the wavelengths of the peaks of the IR radiation and, secondly, the pre-determined characteristic wavelengths of the peaks of the reference curve.

27. The measuring device according to claim 26, wherein the pre-determined characteristic wavelengths correspond to wavelengths of D-glucose absorption peaks.

28. The measuring device according to claim 21, wherein
a) a modulated signal is emitted by the at least one excitation source, and
b) the evaluation unit is configured to determine a dispersion angle, depending on the modulated signal.

29. The measuring device according to claim 16, wherein the sensor apparatus comprises an IR photodiode which detects an IR radiation which is stimulated in the body surface by the electromagnetic radiation coupled-in to the body surface, in order to form a reference signal in order to correct a temperature-related variation of the IR signal detected at the plurality of measurement positions.

30. The measuring device according claim 16, wherein the coupling-in apparatus comprises a measuring head, a form of which is configured to receive at least one of a lower fingertip, an upper fingertip, a heel and an ear lobe of the test object.

31. The measuring device according claim 30, which is configured to do at least one of:
a) determining, before executing a measuring procedure, whether at least one of a lower fingertip, an upper fingertip, a heel and an ear lobe of the test object is positioned in a pre-determined region on the measuring head, or
b) coupling the electromagnetic radiation emitted by the at least one excitation source areally into the body surface with an optical fiber bundle or an optical unit.

32. The measuring device according to claim 16, wherein the electromagnetic radiation generated by the at least one excitation source lies in a range of 250 nm to 30000 nm.

33. The measuring device according to claim 17, wherein the at least one excitation source is a tuneable excitation source which can be tuned through a pre-determined spectral range which comprises one or more peaks in a D-glucose absorption band.

34. The measuring device according to claim 33, wherein the D-glucose absorption band is a D-glucose absorption band in an IR range.

35. The measuring device according to claim 17, wherein the at least one excitation source is a tuneable quantum cascade laser, wherein the electromagnetic radiation generated lies in a range from 1 μm to 30 μm.

36. The measuring device according to claim 35, wherein the electromagnetic radiation generated lies in a range from 7 μm to 14 μm.

37. The measuring device according to claim 21, wherein the blood parameter value is a D-glucose concentration.

38. The measuring device according to claim 19, wherein the IR area sensor is an IR CCD sensor.

39. The measuring device according to claim 16, configured for non-invasive determination of a D-glucose concentration.

40. A method for measuring raw data for determining a blood parameter, comprising performing the following steps with a measuring device according to claim 16:
a) generating electromagnetic radiation,
b) coupling the electromagnetic radiation into a body surface of a measurement object, and
c) detecting an IR radiation which is stimulated in the body surface by the electromagnetic radiation coupled-in to the body surface,
wherein the electromagnetic radiation is coupled areally into the body surface at a plurality of measurement positions.

41. A method for measuring raw data for determining a blood parameter, comprising the steps:
a) generating electromagnetic radiation,
b) coupling the electromagnetic radiation into a body surface of a measurement object, and
c) detecting an IR radiation which is stimulated in the body surface by the electromagnetic radiation coupled-in to the body surface,
wherein the electromagnetic radiation is coupled areally into the body surface at a plurality of measurement positions.

42. The measuring method according to claim 41, wherein the electromagnetic radiation coupled-in to the body surface is tuned during a measuring procedure across a pre-determined spectral range in at least one of a visible or an IR range.

43. The measuring method according to claim 41, wherein a D-glucose concentration is determined non-invasively.
